(19) United States
(12) Patent Application Publication
(10) Pub. No.: US 2004/0161473 A1
(43) Pub. Date: Aug. 19, 2004

(54) Beneficial materials for topical or internal use by a human or other animal

(75) Inventor: Ashok V. Joshi, Salt Lake Cotu, UT (US)

Correspondence Address: FACTOR & LAKE, LTD, 1327 W. WASHINGTON BLVD., SUITE 5G/H, CHICAGO, IL, 60607, US

(73) Assignee: Ashock V Joshi, Salt Lake City, UT (US)

(21) Appl. No.: 10/708,036

(22) Filed: Feb. 4, 2004

Related U.S. Application Data
(63) Continuation of application No. 09/900,480, filed on Jul. 6, 2001, now abandoned.

(51) Int. Cl. A61K 33/40

(52) U.S. Cl. 424/613; 424/423

(57) ABSTRACT

A beneficial material for medical application in association with a substrate comprises a support material and a reactive material. The reactive material is ion exchanged into the support material. The medical application may comprise a wound healing device, insect control adhesives and anti-microbial creams. The invention further discloses other beneficial materials, such as ionically conductive compounds, photoactive compounds and materials for medical application and methods of formulating same.

### CROSS REFERENCE TO RELATED APPLICATIONS

This is a continuation of co-pending U.S. Application Ser. No. 09/900,480, filed Jul. 6, 2001.

### BACKGROUND OF INVENTION

1. Field of the Invention

The invention relates in general to beneficial materials, and in particular to a beneficial material which is coated upon, or impregnated within a medically related device for use internally or topically by humans or other animals. The beneficial material includes anti-microbial and antibacterial properties. In particular, the material is intended to kill or neutralize contaminants, such as micro-organisms, germs, bacteria, viruses, undesirable chemicals and/or compounds, etc.

2. Background Art

The need to kill and/or neutralize contaminants and insects on humans and other animals has long been necessary to ward off infections and diseases. While the use of materials which have anti-microbial, anti-bacterial and insecticidal properties has been known, the efficiency and efficacy of their administration remains problematic for various reasons. For example, certain topical materials have been developed which have anti-microbial and anti-bacterial properties. The application of such topical materials has been problematic as the anti-microbial, anti-bacterial and insecticidal properties often wear off quickly. Other materials are relatively highly toxic to humans and to other animals upon which they are applied. Yet, other materials, while having some efficacy when applied topically, cannot be applied internally to the body of humans and animals for various reasons. Further, certain of these materials have poor performance and must be applied in heavy doses.

It is therefore an object of the invention to provide a material for use on humans and other animals which exhibit anti-bacterial, anti-microbial as well as insecticidal properties.

It is another object of the invention to provide a material which can be used topically or internally as an anti-bacterial, anti-microbial or insecticidal material.

These and other objects will become apparent in light of the present specification, claims and drawings.

### SUMMARY OF INVENTION

The invention comprises a beneficial material for medical application in association with a substrate including a support material and a reactive material. The reactive material is ion exchanged into the support material and the support material is associatable with the substrate.

In one embodiment, the support material comprises any one of ionomers, anion exchange membranes, cation exchange membranes, Nasicon and Nafion.

In another embodiment, the reactive material comprises any one of noble metals, peroxides and halogens.

In such embodiments, the substrate may comprise a formulation in a paste, putty, epoxy spray, tar or membrane form for topical application, wound healing devices, prosthetic devices and other implantable devices.

In another aspect of the invention, the invention comprises a beneficial material for medical application in association with a substrate. The beneficial material comprises an ionically conductive compound associatable with the substrate.

In a preferred embodiment, the ionically conductive compound comprises a halide of a noble metal in selective combination with a high surface area metal oxide, i.e. a metal oxide having a surface area greater than 3 m²/gm.

In one embodiment, the noble metal comprises one of the group consisting of: Ag, Au, Pt, Cu, Pd, Rh, Ir and Ru. In one such embodiment, the metal of the metal oxide comprises one of the group consisting of Ag, Au, Pt, Cu, Al, Ti, Si, Pd, Rh, Ir, Ru, Zn, Sn and Mg.

In another such embodiment, the substrate may comprise a formulation in a paste, putty, epoxy spray or tar form for topical application, wound healing devices, prosthetic devices and other implantable devices.

In another aspect of the invention, the invention comprises a beneficial material for medical application in association with a substrate. The material comprises a photoactive compound associatable with the substrate.

In one such embodiment, the photoactive compound comprises one of the group consisting of combinations of compounds including TiO₂ and Titanates, Fe₂O₃ and compounds of Fe₂O₃ and other oxides, Silver and Copper Oxides, halides and chalcogenides, Vanadium pentoxide and vandates, Tin oxides and stannates, Silver Ion Conductors, NbO₂ and Niobates, TiO₂ and NbO₂ solid solutions, Bi₂O₃ and bismuth chalcogenides, Silicon and Germanium doped with p-type and n-type impurities, P-N junctions of semi-conductors, such as Si, ZnS, GaAs, etc., Photovoltaic materials, such as silicon, Ge, InP, ZnP, Zinc chalcogenides and Zn oxides and Zn phosphides.

The invention may likewise be directed to a wound healing device. In particular, the wound healing device includes a substrate and a beneficial material. The substrate is capable of association with a wound of a human or other animal. The beneficial material is water insoluble peroxide associated with the substrate. The beneficial material comprises one of an ionically conductive compound, a photoactive compound and a reactive material ion-exchanged with a support material. Preferably, the substrate comprises one of a woven pad and a gauze pad.

In an embodiment of the wound healing device wherein the beneficial material comprises an ion exchange material, the reactive material comprises one of the group consisting of: ionomers, anion exchange membranes, cation exchange membranes, Nasicon and Nafion and the reactive material comprises one of the group consisting of: noble metals, metals, halogens and photoactive compounds comprising combinations of compounds including TiO₂ and Titanates, Fe₂O₃ and compounds of Fe₂O₃ and other oxides, Silver and Copper Oxides, halides and chalcogenides, Vanadium pentoxide and vandates, Tin oxides and stannates, Silver Ion Conductors, NbO₂ and Niobates, TiO₂ and NbO₂ solid solutions, Bi₂O₃ and bismuth chalcogenides, Silicon and Germanium doped with p-type and n-type impurities, P-N junctions of semiconductors, such as Si, ZnS, GaAs, etc., Photovoltaic materials, such as silicon, Ge, InP, ZnP, Zinc chalcogenides and Zn oxides and Zn phosphides.

In embodiments of the wound healing device having an ionically conductive compound, the compound may comprise an halide of a noble metal in selective combination with a metal oxide. In other wound healing device embodiments having a photoactive compound, the photoactive compound may comprise combinations of compounds including TiO₂ and Titanates, Fe₂O₃ and compounds of Fe₂O₃ and other oxides, Silver and Copper Oxides, halides and chalcogenides, Vanadium pentoxide and vandates, Tin oxides and stannates, Silver Ion Conductors, NbO₂ and Niobates, TiO₂ and NbO₂ solid solutions, Bi₂O₃ and bismuth chalcogenides, Silicon and Germanium doped with p-type and n-type impurities, P-N junctions of semiconductors, such as Si, ZnS, GaAs, etc., Photovoltaic materials, such as silicon, Ge, InP, ZnP, Zinc chalcogenides and Zn oxides and Zn phosphides.

In yet another wound healing device embodiments having a water insoluble peroxide compound, the water insoluble and slow reactive peroxide compound comprises one or more of the combinations of peroxides of Mg, Ca, Ba, Ag, Cu, Pt, Au, Sn, Zn, Ru, Ir, among others.

In another embodiment, the reactive material may comprise water insoluble excess oxygen containing compounds such as perovskites of La₂NiO₄+δ, La₂CuO₄+δ, CeNiO₄+δ and Ce₂CuO₄+δ.

The invention further comprises a method of incorporating a beneficial material to a substrate. The method comprises the steps of providing a substrate, coating the substrate with a support material and ion exchanging a reactive material with the support material.

In a preferred embodiment, the step of coating comprises one of the following steps: spraying the substrate with a support material, painting the substrate with a support material and dipping the substrate into a support material.

In another aspect of the invention, the invention comprises a method of incorporating a beneficial material to a fluid or semi-solid substrate. The method comprises the steps of providing a fluid or semi solid substrate, providing the beneficial material and mixing the beneficial material within the substrate. In such an embodiment, the beneficial material comprises one of an ionically conductive compound, a photoactive compound a reactive material ion-exchanged with a support material and water insoluble, slowly reactive metal peroxide materials.

In one such embodiment of the method, the method further includes the step of granulating the beneficial material.

In another aspect of the invention, the invention may comprise a method of associating a beneficial material with a substrate. The method comprises the steps of providing a substrate, providing beneficial material, mixing the beneficial material within the substrate; and molding the mixed beneficial material and substrate into a desired configuration. In such an embodiment, the beneficial material comprises one of an ionically conductive compound, a photoactive compound and a reactive material ion-exchanged with a support material.

In one embodiment of the method, the method further includes the step of granulating the beneficial material.

In another aspect of the invention, the invention comprises a method of applying a beneficial material to a human or other animal. The method first includes the step of providing a beneficial material, wherein the beneficial material comprises one of an ionically conductive compound, a photoactive compound, an organic insecticide material and a reactive material ion-exchanged with a support material. Subsequently, the material is associated with a substrate, wherein the substrate comprises a fluid or semisolid material. Subsequently, the combined beneficial material and substrate is applied upon the body of a human or animal.

In one embodiment, the step of applying comprises one of the steps of coating, painting, or pouring of the combined beneficial material and substrate upon the body of a human.

### BRIEF DESCRIPTION OF DRAWINGS

FIG. 1 of the drawings is an embodiment of a beneficial material, showing, in particular, application of same upon an implantable device (i.e. a hip);

FIG. 2 of the drawings is an embodiment of a beneficial material, showing, in particular, application of same in a liquid/paste/epoxy form poured topically onto an animal;

FIG. 3 of the drawings is an embodiment of a beneficial material, showing, in particular, application of same in a wound healing device (i.e. a bandage);

FIG. 4 of the drawings is an embodiment of a beneficial material, showing, in particular, application of same in an prosthetic device (i.e. artificial leg); and

FIG. 5 of the drawings is an embodiment of a beneficial material showing, in particular, application of same in a wound healing device (noble metal or halogen ion exchange membrane).

### DETAILED DESCRIPTION

While this invention is susceptible of embodiment in many different forms, there is shown in the drawings and will be described in detail, several specific embodiments with the understanding that the present disclosure is to be considered as an exemplification of the principles of the invention and is not intended to limit the invention to the embodiments illustrated.

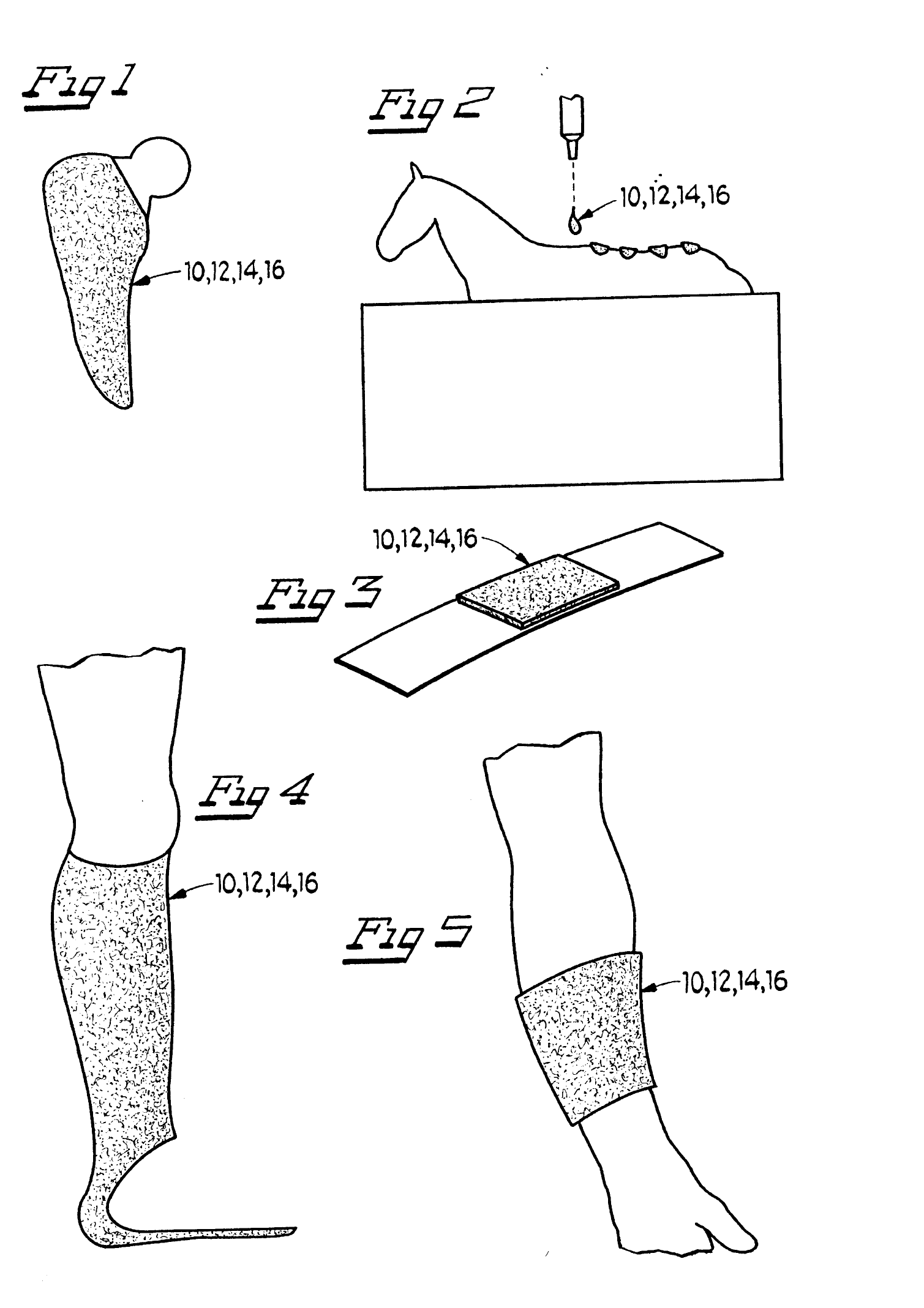

Referring now to the Figures, and in particular to FIG. 1, beneficial material 10 is shown as being applied to substrate 12 As will be explained below, substrate 12 may comprise a variety of devices or materials for use in association with various animals, including, but not limited to humans, domesticated animals, birds, fish, etc, to name just a few. These devices may be used topically as well as within the body (i.e. the devices may be implantable).

In the embodiment shown in FIG. 1, substrate 12 comprises an artificial hip. In such an embodiment, substrate 12 may comprise a variety of materials, including but not limited to titanium, stainless steel, ceramics, composites, to name a few. Such an artificial hip is intended for insertion into the body for a plurality of years to replace a hip joint which has deteriorated. In other embodiments, substrate 12 may comprise insecticide formulations (in a paste, putty, epoxy spray or tar form) for topical application, i.e. a fluid or semi-solid of varying viscosity (FIG. 2), wound healing devices, i.e. bandages (FIG. 3), prosthetic devices, i.e. limbs, eyes, etc. (FIG. 4) and other implantable devices.

Referring again to FIG. 1, beneficial material 10 comprises support material 14 and reactive material 16. Support material 14 may comprise a variety of materials, including, but not limited to anion exchange membranes, cation exchange membranes, Nafion, Nasicon, as well as other ionomers. Reactive material 16 may comprise a variety of different materials, including, but not limited to noble metal compounds (i.e. compounds of Ag, Au, Pt, Cu, Al, Pd, Rh, Ir, Ru, among others) as well as halogens (F, Ci, I, Br) which are ion exchanged into the support material. In other embodiments, beneficial material 10 may comprise a cationic membrane (i.e. ion exchanged with a noble metal ion). Examples of the foregoing ion exchanged membranes comprise Cu-Nasicon; Cu-Nafion; Ag-Nasicon; Ag-Nafion; Au-Nasicon; Au-Nafion; I₂-Anion Membranes, Br₂-Anion Membranes, to name a few.

In yet other embodiments, beneficial material 10 may comprise metal oxides (i.e., oxides of Magnesium) as well as noble metal oxides. Examples of the foregoing comprise AgO; Au₂O₃, MgO₂, CuO. In certain embodiments, the metals Ag, Au, Pt, Cu, Al, Pd, Rh, Ir, Ru, Zn, Rb, Mg, Ca, Sn may be selectively combined organic insecticide materials, including but not limited to Deet and Spinosad.

In yet other embodiments, beneficial material 10 may comprise ionically conductive compounds comprising halides of noble metals and other metals selectively along with metal oxides, and preferably with metal oxides having a high surface area, i.e. a surface area greater than 3 m²/gm. Examples of these include Agl+Al₂O₃, Cal+Al₂O₃; Aul+Al₂O; Ag₄Rbl₅+Al₂O₃, among others.

In other embodiments, the reactive material may one of water insoluble peroxides and water insoluble excess oxygen containing compounds which are associated with a substrate. In such an embodiment, the water insoluble peroxides may comprise MgO₂, BaO₂, SnO₂, AgO, CaO₂ and ZnO₂, among others. In other such embodiments, the water insoluble excess oxygen containing compounds may comprise perovskites of La₂NiO₄+δ, La₂CuO₄+δ, Ce-NiO₄+δ and Ce₂CuO₄+δ, among others.

In still other embodiments, beneficial material may comprise photoactive compounds such as those listed on Table 1, below. Such compounds have effects in the presence of light. For example, TiO₂, upon exposure to UV light, and, in the presence of humidity in the air, releases hydroxyl ions which is repellant of insects. The other example comprises a photovoltaic material which upon exposure to sunlight produce voltage, and, in turn, repel insects and microbial activity.

TABLE I
PHOTOCATALYTIC MATERIALS

TiO₂ and Titanates
Fe₂O₃ and compounds of Fe₂0₃ and other oxides
Silver and Copper Oxides, halides and chalcogenides
Vanadium pentoxide and vandates
Tin oxides and stannates
Silver Ion Conductors
NbO₂ and Niobates
TiO₂ and NbO2 solid solutions
Bi₂O₃ and bismuth chalcogenides
Silicon and Germanium doped with p-type and n-type impurities
P-N junctions of semiconductors, such as Si, ZnS, GaAs, etc.
Photovoltaic materials, such as silicon, Ge. InP, ZnP
Zinc chalcogenides and Zn oxides and Zn phosphides.

As will be explained, in each embodiment of the invention, beneficial material 12 has properties which tend to kill or neutralize contaminants, such as microorganisms, germs, insects bacteria, viruses, undesirable chemicals and/or compounds, etc.

Various different manners in which to apply beneficial material have been contemplated. By way of example, a device or material can be coated (sprayed, dipped, painted, etc.) with beneficial material. In other embodiments, the beneficial material may be granulated and introduced into a fluid, semi-solid or solid material. In another group of embodiments particularly well suited for use in association with moldable (i.e. plaster, plastic, composite, etc.) substrates, the beneficial agent may be introduced (in either a granulated powdered or ungranulated configuration) into the substrate. Once introduced, the mixture of beneficial agent and substrate can be molded into a desired configuration. For example, a plaster of paris material can be mixed with one such beneficial agent powder or solution before it is made.

For example, relative to the embodiment of FIG. 1, beneficial material 12 may comprise Ag (reactive material 16) which has been ion exchanged into Nafion (as substrate 14) to form Ag-Nafion material. In such an embodiment, the Nafion substrate may be dip coated upon the device and, subsequently, the device with the coating may be placed into solution containing Ag ions for ion exchange. When a protonated Nafion membrane is dipped into silver nitrate solution, Ag ions in the solution ion exchange with protons in the Nafion, thereby rendering an Ag-Nafion membrane.

In the embodiment shown in FIG. 2 which comprises a topically applied fluid or semi-solid material (such as adhesives, epoxies, etc), a beneficial material may comprise materials chosen from Table I or noble metal ionomers which is ground and introduced into a paste-like substrate such as epoxies, adhesives, glue or tars. The paste can then be applied onto the surface of cattle or to another animal to kill or neutralize contaminants and insects over a prolonged period of time.

In the embodiment of FIG. 3, the beneficial material may be associated with a wound healing device, such as a bandage. In particular, the bandage may include a substrate (i.e. woven pad or gauze with an adhesive to attach to the skin) and a beneficial material which is applied to the substrate. In one embodiment, the beneficial material may coated upon the substrate, or alternatively, the fibers which are used to form the substrate may be treated prior to the manufacture of the fibers into the substrate. In yet other embodiments, the beneficial material may comprise a fluid or a semi-solid material which is applied to the substrate prior to or in conjunction with its use. In yet another embodiment, the cation exchanged membrane with noble metal cations is effective as a bandage for wound healing.

In operation of the embodiment shown in FIG. 3, as the wound healing device (associated with the beneficial material) is positioned to cover a wound, the beneficial material is in close proximity to a wound. In turn, the beneficial material kills or otherwise neutralizes contaminants. This, in turn, precludes infection of the wound.

In the embodiment shown in FIG. 4 which may comprise a prosthetic device, beneficial material may be powdered or granulated and introduced into a substrate which may comprise a moldable plastic or resin material. In such an embodiment, the materials are mixed and the resulting mixture may be cast or molded into the desired form.

Of course, other methods of applying the beneficial material are contemplated such as introducing the beneficial material into a liquid material (such as a paint), wherein the resulting liquid material can be applied onto a device (painted or sprayed).

Several examples were developed and tested using different formulations of beneficial material. The tests are merely examples to exemplify the principles of the invention, and it will be understood that the beneficial material, or the application of same is not limited to these examples.

### EXAMPLE 1

To formulate the material in this example, a circular sample of Sodium Nafion (DuPont material) was first cut with a punch. The sample was then cleaned with distilled water. The Nafion sample was then exposed to a 1 M AgNO₃ solution at 100° C. for three hours and then cooled to room temperature. As a result of the exposure, Ag was ion exchanged into the Nafion. The ion exchanged Nafion samples were then rinsed in distilled water to wash nitrates. The silver exchanged Nafion called Ag-Nafion was now in condition for testing.

### EXAMPLE 2

To formulate the material in this example, a similar circular sample of Nafion was cut with a punch, and the sample was cleaned with distilled water. The Nafion sample was then exposed to a 1 M Cu(NO₃)₂ solution at 100° C. for three hours and then cooled to room temperature. As a result, the Cu was ion exchanged into the Nafion. The ion exchanged Nafion samples were then rinsed in distilled water to wash nitrates. The copper exchanged Nafion called Cu-Nafion was now in condition for testing.

### EXAMPLE 3

To formulate the material in this example, a similar circular sample of Nafion was cut with a punch and cleaned with distilled water. The Nafion sample was then coated with an Au/Pd coating which was sputter deposited at 60 mA current and 200 Mtorr. The coated Nafion was then placed into a 0.25 M HNO₃ solution for three hours at 40° C. In this manner, the solution first dissolved the Au/Pd coating then the coating was ion-exchanged into the Nafion. The Au/Pd ion exchanged Nafion was now in condition for testing.

### EXAMPLE 4

To formulate the material in this example, a similar circular sample of Nasicon was placed in a 1M AgNO₃ solution at 50° C. for three hours and cooled to room temperature. As a result, the Ag was ion exchanged into the Nasicon. Subsequently, the ion exchanged samples were rinsed in distilled water to remove any residual nitrate material. The Ag-Nasicon was then tested for its anti-microbial properties, wherein an Ag-Nasicon pellet was formed for testing.

### EXAMPLE 5

To formulate the material in this example, coated steel rods were prepared. Specifically, 441 stainless steel rods were chemically etched in HNO₃/H₃PO₄/Acetic acid/H₂O solution for one hour. The samples were then washed. Next, the samples were then dip coated in five separate coats of Nafion solution obtained from DuPont. Each coating was performed at 100° C. Once coated with the Nafion solution, the steel samples were placed in a 1 M AgNO₃ solution at 40° C. for three hours. The samples were then cooled to room temperature and washed in distilled water to remove any nitrates. These steel rods were then tested for their antimicrobial properties.

### EXAMPLE 6

To formulate the material in this example, coated steel rods were prepared. Specifically, 441 stainless steel rods were chemically etched in HNO₃/H₃PO₄/Acetic acid/H₂O solution for one hour. The samples were then washed. Next, a slurry of an Ag ion exchanged in a Nasicon ceramic was prepared. The slurry was spray coated upon the treated steel rods. Subsequently, the slurry coated rods were fired at 850° C. for 1 hour. Thus, these steel rods coated with Ag ion exchanged Nasicon were tested for their anti-microbial activity.

### EXAMPLE 7

To formulate the material in this example, Agl and Al₂O₃ at 70/30 wt % were aggressively mixed with CH 40H (methanol) for 15 minutes in a paint shaker. They were subsequently dried and calcined at 560° C. for 2 hours into a powder. The formulation was then pressed into 1 inch disks at 35 Kpsi.

### EXAMPLE 8

To formulate the material in this example, MgO₂ powder was pressed into 1 inch ceramic disks. The MgO₂ was not pre-milled, and no binder was added prior to pressing. This ceramic MgO₂ disk was then tested for its antimicrobial activity.

The foregoing formulations were tested to determine effectiveness relative to killing or neutralizing contaminants, such as microorganisms, germs, bacteria, viruses, undesirable chemicals and/or compounds, etc. To test the formulations, the prepared samples were placed in a 20 ml beaker. Next, a 5 ml 10⁻⁴ dilution stationary phase culture of E coli was poured into each of the beaker. After three hours of testing the sample under room temperature, 0.2 ml of the tested culture of each candidate was placed in a petri dish. The petri dishes were placed into an oven for 14 hours at 37° C. The positive control was an untreated 10⁻⁴ dilution of stationary phase culture and the negative control was an empty petri dish.

The results were as follows:

| Exp. Results | Example 1 | Example 2 | Example 3 | Example 4 | Control |
|---|---|---|---|---|---|
| No. of | 0 | 0 | 0 | 0 | 440 |
| Colonies | | | | | |
| % of Colonies | 100% | 100% | 100% | 100% | 0% |
| Killed | | | | | |
| Exp. Results | Example 5 | Example 6 | Example 7 | Example 8 | Control |
| No. of | 0 | 0 | 0 | 0 | 440 |
| Colonies | | | | | |
| % of Colonies | 100% | 100% | 100% | 100% | 0% |
| Killed | | | | | |

As can be seen from the results above, each of the examples provided excellent anti-bacterial properties.

The foregoing description merely explains and illustrates the invention and the invention is not limited thereto except insofar as the appended claims are so limited, as those skilled in the art who have the disclosure before them will be able to make modifications without departing from the scope of the invention.

1. A beneficial material for medical application in association with a substrate comprising:
a support material; and
a reactive material associated with the support material,
wherein the reactive material is selected from the group comprising water insoluble peroxides and water insoluble excess oxygen containing compounds.

2. The beneficial material of claim 1 wherein the support material comprises one of the group consisting of:
ionomers, anion exchange membranes, cation exchange membranes, Nasicon and Nafion.

3. The beneficial material of claim 1 wherein a substrate may comprise one of the group consisting of: formulations in a paste, putty, epoxy, adhesive, glue, spray or tar form for topical application, wound healing devices, prosthetic devices and other implantable devices.

4. The beneficial material of claim 1 wherein the water insoluble peroxides comprise one of the group consisting of: MgO 2, BaO₂, SnO₂, AgO, CaO₂, CuO₂ and ZnO₂.

5. The beneficial material of claim 1 wherein the water insoluble excess oxygen containing compounds comprise one of the group consisting of perovskites of La₂NiO₄+δ, La₂CuO₄+δ, CeNiO₄+δ and Ce₂CuO₄+δ.

6. A wound healing device comprising:
a substrate capable of association with a wound of a human or other animal; and
a reactive material associated with the substrate,
wherein the reactive material is selected from the group comprising comprises water insoluble peroxides and water insoluble excess oxygen containing compounds.

7. The wound healing device of claim 6 wherein the substrate comprises one of a woven pad and a gauze pad.

8. A method of incorporating a beneficial material to a fluid or semi-solid substrate comprising the steps of:
providing a fluid or semi solid substrate;
providing the beneficial material; and
mixing the beneficial material within the substrate,
wherein the beneficial material is selected from the group comprising water insoluble peroxides and water insoluble excess oxygen containing compounds.

9. The method of claim 8 further comprising the step of granulating the beneficial material.

10. The method of claim 9 wherein the substrate may comprise one of the group consisting of paint, epoxy, adhesive, glue and tar.
